(19) United States
(12) Patent Application Publication
(10) Pub. No.: US 2024/0423952 A1
(43) Pub. Date: Dec. 26, 2024

(54) COMPOSITIONS AND METHODS FOR TREATING CONGENITAL DISORDERS

(71) Applicants: Ibagen Pharmaceuticals, Inc., Lexington, MA (US); Mount Sinai Hospital, Toronto (CA)

(72) Inventors: Hanlan Liu, Lexington, MA (US); Mei Zhen, Toronto (CA); Claudia Arndt, Windsor (CA); Yibin Xiang, Dracut, MA (US); Jianming Yin, Lexington, MA (US); Wesley Hung, Toronto (CA)

(21) Appl. No.: 18/708,315

(22) PCT Filed: Nov. 12, 2022
(86) PCT No.: PCT/US22/49743

§ 371 Date: May 8, 2024

Related U.S. Application Data
(60) Provisional application No. 63/279,230, filed on Nov. 15, 2021.

(51) Int. Cl. A61K 31/395; A61K 31/138; A61K 31/245; A61K 31/277; A61K 31/4418; A61K 31/4745

(52) U.S. Cl. CPC A61K 31/395; A61K 31/138; A61K 31/245; A61K 31/277; A61K 31/4418; A61K 31/4745

(57) ABSTRACT

The invention provides novel pharmaceutical compositions and methods thereof for therapeutic use in treating various congenital diseases and conditions, e.g., Congenital Contractures of the Limbs and Face, Hypotonia, and Developmental Delay (CLIFAHDD), and related diseases and conditions.

### PRIORITY CLAIMS AND RELATED PATENT APPLICATIONS

This application claims the benefit of priority to U.S. Provisional Application Ser. No. 63/279,230, filed Nov. 15, 2021, the entire content of which is incorporated herein by reference.

### TECHNICAL FIELD OF THE INVENTION

The invention generally relates to pharmaceutical compositions and therapeutic methods. More particularly, the invention provides novel pharmaceutical compositions and methods thereof for therapeutic use in treating various congenital diseases and conditions, e.g., Congenital Contractures of the Limbs and Face, Hypotonia, and Developmental Delay (CLIFAHDD), and related diseases and conditions.

### BACKGROUND OF THE INVENTION

CLIFAHDD is a recently established, emerging neurodevelopmental disorder characterized by congenital contractures of the limbs and face, resulting in characteristic facial features, hypotonia, and variable degrees of developmental delay. CLIFAHDD cases were first reported in 2015. Three out of 5 individuals with CLIFAHDD condition had resulted in early death. This is an emerging neurodevelopmental disorder genetic hotspot with no apparent specificity in ethnic backgrounds including Caucasian, Japanese, and Arabian. (Chong, et al 2015 Am J Hum Genet. 96, 462-473; Fukai, et al. 2016 J Hum Genet 61, 451-455; Gal, et al. 2016 Euro J Med Genet 59, 204-209; Karakaya, et al. 2016 Neuropediatrcis 47 (4), 273-277; Lozic, et al. 2016 Annals of Clinical and Translational Neurology, 876-883; Sivaraman, et al. 2016 J Clin Neur 34, 222-223; Wang, et al. 2016 Clin Genet 90, 556-557.)

Genetic studies have been performed in search of human diseases associated with the sodium leak channel nonselective (NALCN) variants. Exome sequencing identified missense mutations in NALCN in four families affected by CLIFAHDD syndrome. NALCN mutation represented 0.11% in a series of 4385 individuals with neurodevelopmental disabilities. NALCN-related syndrome has been classified as the CLIFAHDD. Importantly, some symptoms of the syndrome, such as intellectual disability and developmental delay, are shared by mutations that could lead to either loss or gain of the channel activity. Dominant, gain-of-function mutations in NALCN can cause neuro-developmental phenotypes that partially overlap with, while being mostly distinct from that associated with recessive mutations in the NALCN gene. This notion has now been confirmed in two other patient cohorts. NALCN R1181Q is the only known recurring, de novo missense mutation from multiple patient cohorts. (Al-Sayed, et al. 2013 Am J Hum Genet 93, 721-726; Bend, et al. 2016 Neurology 87, 1131-1139.)

Disease manifestation of CLIFAHDD occurs as early as within days after birth. It is typically diagnosed through combined clinical features: contractured limbs and faces (most commonly in the form of arthrogryposis), hypotonia, developmental delay, fMRI, and intellectual disability. Patients that are suspected of CLIFAHDD are typically confirmed via genome exon sequencing of the proband, parents and optionally, the unaffected siblings for missense, heterozygous, or biallelic mutations in NALCN, UNC-80, UNC-79 and mNLF. Patients are followed monthly for clinical features. At the present time, there is no approved treatment for CLIFAHDD.

Thus, there is an urgent need for novel therapeutics and treatment methods for congenital diseases and conditions with superior efficacy and safety profiles that provide improved clinical outcomes.

### BRIEF DESCRIPTION OF THE DRAWINGS

FIG. 1. Exemplary results of target response studies using nefopam.

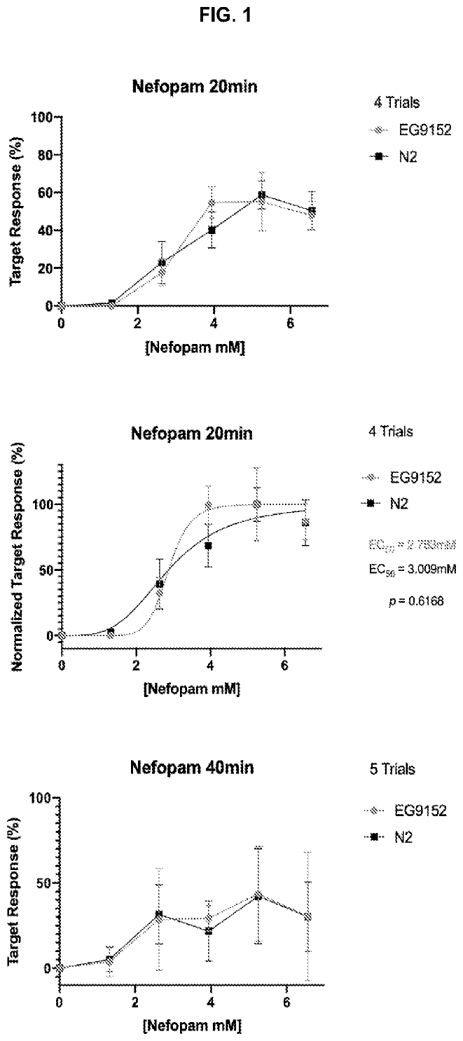

FIG. 2. Exemplary results of target response studies using amlodipine.

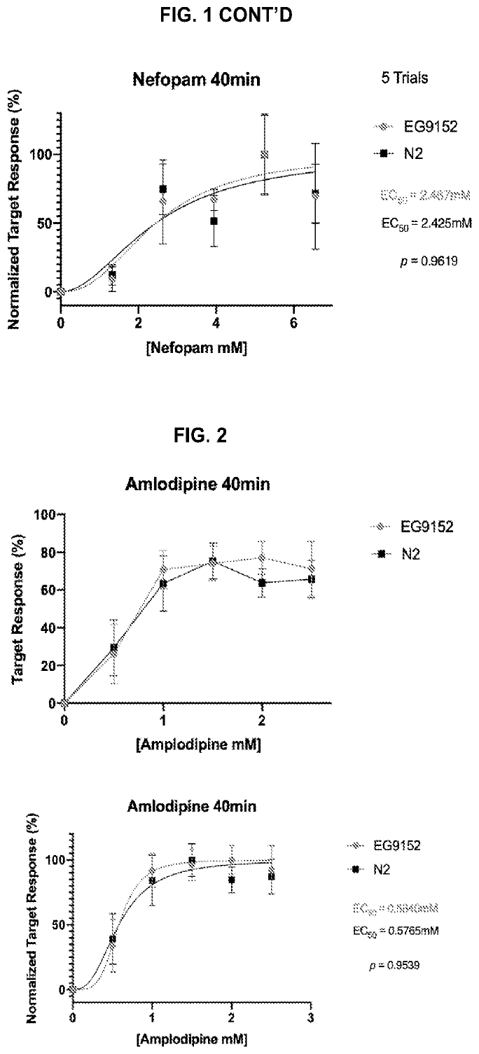

FIG. 3. Exemplary results of target response studies using verapamil.

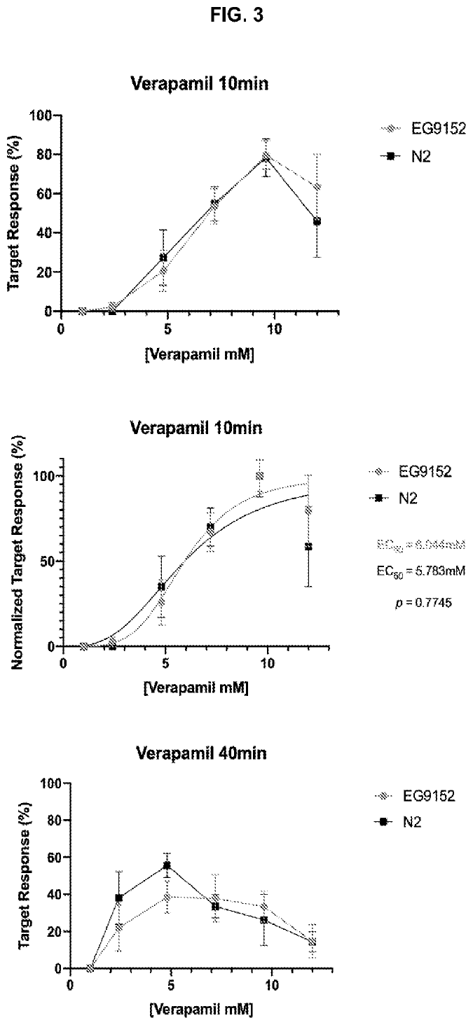

FIG. 4. Exemplary results of leak currents: holding at −60 mV.

FIG. 5. Exemplary results of resting membrane potential: holding at 0 pA.

FIG. 6. Exemplary results of AP firing: quantified by the AP frequency.

FIG. 7. Exemplary results of outward K′ currents: ramp −60˜+80 mV, quantified at +80 mV.

FIG. 8. Exemplary results of amlodipine blocked Outward K⁺ currents.

FIG. 9. Exemplary results of nefopam blocked Outward K⁺ currents.

FIG. 10. Exemplary results of inward Na/Ca currents: quantified by the peak inward current.

FIG. 11. Exemplary results of inward Na/Ca currents: quantified by the peak inward current.

FIG. 12. Exemplary results of inward Na/Ca currents: quantified by the peak inward current.

### SUMMARY OF THE INVENTION

The invention is based in part on the unexpected discovery of novel pharmaceutical compositions and therapeutic methods for treating various congenital diseases and conditions, in particular, CLIFAHDD.

In one aspect, the invention generally relates to a method for treating or reducing a congenital disorder. The method comprises administering to a subject in need thereof a pharmaceutical composition comprising a compound selected from the group consisting of:

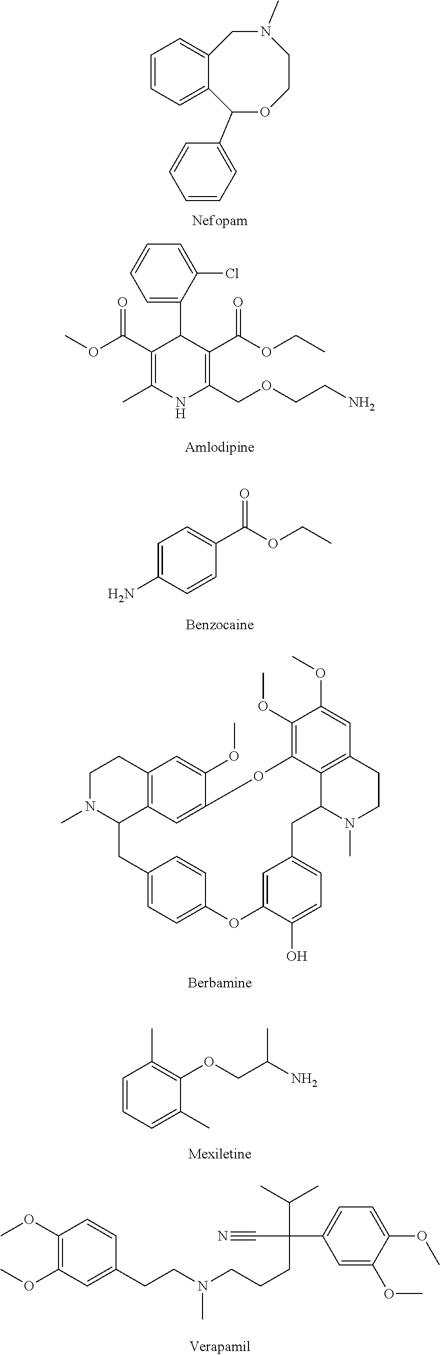

or a pharmaceutically acceptable form (e.g., salt) or an isotope derivative thereof, and a pharmaceutically acceptable excipient, carrier, or diluent, effective to treat or reduce a congenital disorder, or a related disease or condition, in a mammal, including a human.

In another aspect, the invention generally relates to a method for treating or reducing one or more symptoms or diseases associated with NALCN caused by any Gain-of-Function or Loss-of Function gene mutations. The method comprises administering to a subject in need thereof a pharmaceutical composition comprising a compound selected from the heretofore listed compounds, or a pharmaceutically acceptable form (e.g., salt) or an isotope derivative thereof, and a pharmaceutically acceptable excipient, carrier, or diluent, effective to treat or reduce one or more such symptoms, in a mammal, including a human.

In yet another aspect, the invention generally relates to a method for treating or reducing one or more symptoms or diseases associated with NALCN R1181Q mutation. The method comprises administering to a subject in need thereof a pharmaceutical composition comprising a compound selected from the heretofore listed compounds, or a pharmaceutically acceptable form (e.g., salt) or an isotope derivative thereof, and a pharmaceutically acceptable excipient, carrier, or diluent, effective to treat or reduce one or more such symptoms, in a mammal, including a human.

In yet another aspect, the invention generally relates to a method for treating or reducing CLIFAHDD or other related neurological diseases that have overlapping symptoms to CLIFAHDD. The method comprises administering to a subject in need thereof a pharmaceutical composition comprising a compound selected from the heretofore listed compounds, or a pharmaceutically acceptable form (e.g., salt) or an isotope derivative thereof, and a pharmaceutically acceptable excipient, carrier, or diluent, effective to treat or reduce CLIFAHDD, or a related disease or condition, in a mammal, including a human.

In yet another aspect, the invention generally relates to a method for modifying a neuronal membrane property to achieve a change in its activity. The method comprises administering to a subject in need thereof a pharmaceutical composition comprising a compound selected from the heretofore listed compounds, or a pharmaceutically acceptable form (e.g., salt) or an isotope derivative thereof, and a pharmaceutically acceptable excipient, carrier, or diluent, effective to inhibit a neuronal activity, in a mammal, including a human.

In yet another aspect, the invention generally relates to a pharmaceutical composition comprising a therapeutically effective amount of a compound selected from the heretofore listed compounds, or a pharmaceutically acceptable form (e.g., salt) or an isotope derivative thereof, and a pharmaceutically acceptable excipient, carrier, or diluent.

In yet another aspect, the invention generally relates to a unit dosage form comprising the pharmaceutical composition of a compound selected from the heretofore listed compounds.

In yet another aspect, the invention generally relates to use of a compound selected from the heretofore listed compounds, or a pharmaceutically acceptable form (e.g., salt) or an isotope derivative thereof, and a pharmaceutically acceptable excipient, carrier, or diluent, in the preparation of a medicament for treating or reducing CLIFAHDD or other related neurological diseases that have overlapping symptoms to CLIFAHDD.

In yet another aspect, the invention generally relates to use of a compound selected from the heretofore listed compounds, or a pharmaceutically acceptable form (e.g., salt) or an isotope derivative thereof, and a pharmaceutically acceptable excipient, carrier, or diluent, in the preparation of a medicament for treating or reducing one or more symptoms or diseases associated with NALCN R1181Q mutation.

In yet another aspect, the invention generally relates to use of a compound selected from the heretofore listed compounds, or a pharmaceutically acceptable form (e.g., salt) or an isotope derivative thereof, and a pharmaceutically acceptable excipient, carrier, or diluent, in the preparation of a medicament for treating or reducing CLIFAHDD and similar channel-mutation-related congenital disorders.

In yet another aspect, the invention generally relates to use of a compound selected from the heretofore listed compounds, or a pharmaceutically acceptable form (e.g., salt) or an isotope derivative thereof, and a pharmaceutically acceptable excipient, carrier, or diluent, in the preparation of a medicament for modifying a neuronal membrane property to achieve a change in its activity.

### Definitions

Unless defined otherwise, all technical and scientific terms used herein have the same meaning as commonly understood by one of ordinary skill in the art to which this invention belongs. The following terms, unless indicated otherwise according to the context wherein the terms are found, are intended to have the following meanings.

As used herein, the term “cell” refers to any prokaryotic, eukaryotic, primary cell or immortalized cell line, any group of such cells as in, a tissue or an organ. Preferably the cells are of mammalian (e.g., human) origin and can be infected by one or more pathogens.

As used herein, the terms “disease” or “disorder” refer to a pathological condition, for example, one that can be identified by symptoms or other identifying factors as diverging from a healthy or a normal state. The term “disease” includes disorders, syndromes, conditions, and injuries. Diseases include, but are not limited to, proliferative, inflammatory, immune, metabolic, infectious, and ischemic diseases.

As used herein, the term “effective amount” of an active agent refers to an amount sufficient to elicit the desired biological response. As will be appreciated by those of ordinary skill in this art, the effective amount of a compound of the invention may vary depending on such factors as the desired biological endpoint, the pharmacokinetics of the compound, the disease being treated, the mode of administration, and the patient.

As used herein, the term “high dosage” is meant at least 5% (e.g., at least 10%, 20%, 50%, 100%, 200%, or even 300%) more than the highest standard recommended dosage of a particular compound for treatment of any human disease or condition.

As used herein, the term “inhibit” refers to any measurable reduction of biological activity. Thus, as used herein, “inhibit” or “inhibition” may be referred to as a percentage of a normal level of activity.

As used herein, the term “low dosage” refers to at least 5% less (e.g., at least 10%, 20%, 50%, 80%, 90%, or even 95%) than the lowest standard recommended dosage of a particular compound formulated for a given route of administration for treatment of any human disease or condition. For example, a low dosage of an agent that is formulated for administration by inhalation will differ from a low dosage of the same agent formulated for oral administration.

As used herein, a “pharmaceutically acceptable form” of a disclosed compound includes, but is not limited to, pharmaceutically acceptable salts, esters, hydrates, solvates, isomers, prodrugs, and isotopically labeled derivatives of disclosed compounds. In one embodiment, a “pharmaceutically acceptable form” includes, but is not limited to, pharmaceutically acceptable salts, esters, isomers, prodrugs and isotopically labeled derivatives of disclosed compounds. In some embodiments, a “pharmaceutically acceptable form” includes, but is not limited to, pharmaceutically acceptable salts, esters, stereoisomers, prodrugs and isotopically labeled derivatives of disclosed compounds.

In certain embodiments, the pharmaceutically acceptable form is a pharmaceutically acceptable salt. As used herein, the term “pharmaceutically acceptable salt” refers to those salts which are, within the scope of sound medical judgment, suitable for use in contact with the tissues of subjects without undue toxicity, irritation, allergic response and the like, and are commensurate with a reasonable benefit/risk ratio. Pharmaceutically acceptable salts are well known in the art. For example, Berge et al. describes pharmaceutically acceptable salts in detail in J. Pharmaceutical Sciences (1977) 66:1-19. Pharmaceutically acceptable salts of the compounds provided herein include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, besylate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. In some embodiments, organic acids from which salts can be derived include, for example, acetic acid, propionic acid, glycolic acid, pyruvic acid, oxalic acid, lactic acid, trifluoracetic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like.

The salts can be prepared in situ during the isolation and purification of the disclosed compounds, or separately, such as by reacting the free base or free acid of a parent compound with a suitable base or acid, respectively. Pharmaceutically acceptable salts derived from appropriate bases include alkali metal, alkaline earth metal, ammonium and N⁺(C₁₋₄alkyl)⁴ salts. Representative alkali or alkaline earth metal salts include sodium, lithium, potassium, calcium, magnesium, iron, zinc, copper, manganese, aluminum, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate. Organic bases from which salts can be derived include, for example, primary, secondary, and tertiary amines, substituted amines, including naturally occurring substituted amines, cyclic amines, basic ion exchange resins, and the like, such as isopropylamine, trimethylamine, diethylamine, triethylamine, tripropylamine, and ethanolamine. In some embodiments, the pharmaceutically acceptable base addition salt can be chosen from ammonium, potassium, sodium, calcium, and magnesium salts.

In certain embodiments, the pharmaceutically acceptable form is a pharmaceutically acceptable ester. As used herein, the term “pharmaceutically acceptable ester” refers to esters that hydrolyze in vivo and include those that break down readily in the human body to leave the parent compound or a salt thereof. Such esters can act as a prodrug as defined herein. Pharmaceutically acceptable esters include, but are not limited to, alkyl, alkenyl, alkynyl, aryl, aralkyl, and cycloalkyl esters of acidic groups, including, but not limited to, carboxylic acids, phosphoric acids, phosphinic acids, sulfinic acids, sulfonic acids and boronic acids. Examples of esters include formates, acetates, propionates, butyrates, acrylates and ethylsuccinates. The esters can be formed with a hydroxy or carboxylic acid group of the parent compound.

In certain embodiments, the pharmaceutically acceptable form is a “solvate” (e.g., a hydrate). As used herein, the term “solvate” refers to compounds that further include a stoichiometric or non-stoichiometric amount of solvent bound by non-covalent intermolecular forces. The solvate can be of a disclosed compound or a pharmaceutically acceptable salt thereof. Where the solvent is water, the solvate is a “hydrate”. Pharmaceutically acceptable solvates and hydrates are complexes that, for example, can include 1 to about 100, or 1 to about 10, or 1 to about 2, about 3 or about 4, solvent or water molecules. It will be understood that the term “compound” as used herein encompasses the compound and solvates of the compound, as well as mixtures thereof.

In certain embodiments, the pharmaceutically acceptable form is a prodrug. As used herein, the term “prodrug” (or “pro-drug”) refers to compounds that are transformed in vivo to yield a disclosed compound or a pharmaceutically acceptable form of the compound. A prodrug can be inactive when administered to a subject, but is converted in vivo to an active compound, for example, by hydrolysis (e.g., hydrolysis in blood). In certain cases, a prodrug has improved physical and/or delivery properties over the parent compound. Prodrugs can increase the bioavailability of the compound when administered to a subject (e.g., by permitting enhanced absorption into the blood following oral administration) or which enhance delivery to a biological compartment of interest (e.g., the brain or lymphatic system) relative to the parent compound. Exemplary prodrugs include derivatives of a disclosed compound with enhanced aqueous solubility or active transport through the gut membrane, relative to the parent compound.

The prodrug compound often offers advantages of solubility, tissue compatibility or delayed release in a mammalian organism (see, e.g., Bundgard, H., Design of Prodrugs (1985), pp. 7-9, 21-24 (Elsevier, Amsterdam). A discussion of prodrugs is provided in Higuchi, T., et al., “Pro-drugs as Novel Delivery Systems,” A.C.S. Symposium Series, Vol. 14, and in Bioreversible Carriers in Drug Design, ed. Edward B. Roche, American Pharmaceutical Association and Pergamon Press, 1987, both of which are incorporated in full by reference herein. Exemplary advantages of a prodrug can include, but are not limited to, its physical properties, such as enhanced water solubility for parenteral administration at physiological pH compared to the parent compound, or it can enhance absorption from the digestive tract, or it can enhance drug stability for long-term storage.

As used herein, the term “pharmaceutically acceptable” excipient, carrier, or diluent refers to a pharmaceutically acceptable material, composition or vehicle, such as a liquid or solid filler, diluent, excipient, solvent or encapsulating material, involved in carrying or transporting the subject pharmaceutical agent from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. Wetting agents, emulsifiers and lubricants, such as sodium lauryl sulfate, magnesium stearate, and polyethylene oxide-polypropylene oxide copolymer as well as coloring agents, release agents, coating agents, sweetening, flavoring and perfuming agents, preservatives and antioxidants can also be present in the compositions.

As used herein, the terms “subject” and “patient” are used interchangeably herein to refer to a living animal (human or non-human). The subject may be a mammal. The terms “mammal” or “mammalian” refer to any animal within the taxonomic classification mammalia. A mammal may be a human or a non-human mammal, for example, dogs, cats, pigs, cows, sheep, goats, horses, rats, and mice. The term “subject” does not preclude individuals that are entirely normal with respect to a disease or condition, or normal in all respects.

As used herein, the term “therapeutically effective amount” refers to the dose of a therapeutic agent or agents sufficient to achieve the intended therapeutic effect with minimal or no undesirable side effects. A therapeutically effective amount can be determined by a skilled physician, e.g., by first administering a low dose of the pharmacological agent(s) and then incrementally increasing the dose until the desired therapeutic effect is achieved with minimal or no undesirable side effects.

As used herein, the terms “treatment” or “treating” a disease or disorder refers to a method of reducing, delaying or ameliorating such a condition, or one or more symptoms of such disease or condition, before or after it has occurred. Treatment may be directed at one or more effects or symptoms of a disease and/or the underlying pathology. The treatment can be any reduction and can be, but is not limited to, the complete ablation of the disease or the symptoms of the disease. As compared with an equivalent untreated control, such reduction or degree of prevention is at least 5%, 10%, 20%, 40%, 50%, 60%, 80%, 90%, 95%, or 100% as measured by any standard technique.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

As used herein, “at least” a specific value is understood to be that value and all values greater than that value.

As used herein, “more than one” is understood as 2, 3, 4, 5, 6, 7, 8, 9, 10, 11, 12, 13, 14, 15, 16, 17, 18, 19, 20, 25, 30, 40, 50, 100, etc., or any value therebetween.

Unless specifically stated or obvious from context, as used herein, the term “or” is understood to be inclusive.

In this specification and the appended claims, the singular forms “a,” “an,” and “the” include plural reference, unless the context clearly dictates otherwise.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein can be modified by the term about.

Any compositions or methods disclosed herein can be combined with one or more of any of the other compositions and methods provided herein.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

### DETAILED DESCRIPTION OF THE INVENTION

The invention provides novel pharmaceutical compositions and therapeutic methods for treating various congenital diseases and conditions, in particular, CLIFAHDD. As disclosed in detail herein, extensive studies were conducted using N2 non-transgenic worms and EG9152 GOF worms and dose curves and electrophysiological studies were performed. Neuronal activities through agonistic effect on NALCN-mediated channel activity and through antagonistic effect on NALCN-mediated channel activity were further evaluated. These and other studies led to the unexpected discovery of compounds with promising potency on modulating neuronal activities and treatment of various congenital diseases and conditions, in particular CLIFAHDD.

In one aspect, the invention generally relates to a method for treating or reducing a congenital disorder. The method comprises administering to a subject in need thereof a pharmaceutical composition comprising a compound selected from Table 1.

TABLE 1

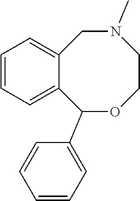 [SMILES: CN1CCOC(c2ccccc2)c2ccccc2C1]

Nefopam

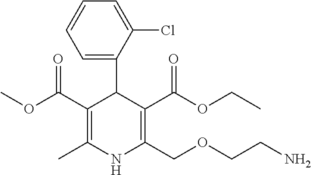 [SMILES: CCOC(=O)C1=C(COCCN)NC(C)=C(C(=O)OC)C1c1ccccc1Cl]

Amlodipine

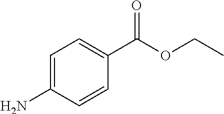

Benzocaine

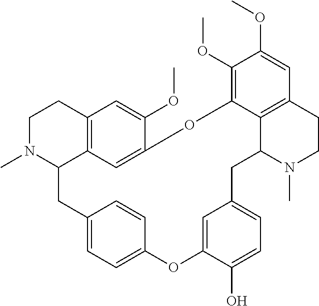 [SMILES: COc1cc2c3cc1Oc1c(OC)c(OC)cc4c1C(Cc1ccc(O)c(c1)Oc1ccc(cc1)CC3N(C)CC2)N(C)CC4]

Berbamine

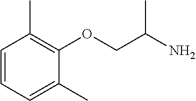

Mexiletine

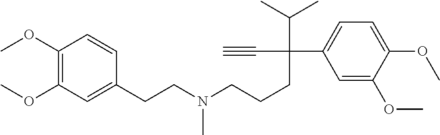

Verapamil

or a pharmaceutically acceptable form (e.g., salt) or an isotope derivative thereof, and a pharmaceutically acceptable excipient, carrier, or diluent, effective to treat or reduce a congenital disorder, or a related disease or condition, in a mammal, including a human.

In another aspect, the invention generally relates to a method for treating or reducing one or more symptoms or diseases associated with NALCN caused by any Gain-of-Function or Loss-of Function gene mutations. The method comprises administering to a subject in need thereof a pharmaceutical composition comprising a compound selected from Table 1, or a pharmaceutically acceptable form (e.g., salt) or an isotope derivative thereof, and a pharmaceutically acceptable excipient, carrier, or diluent, effective to treat or reduce one or more such symptoms, in a mammal, including a human.

In yet another aspect, the invention generally relates to a method for treating or reducing one or more symptoms or diseases associated with NALCN R1181Q mutation. The method comprises administering to a subject in need thereof a pharmaceutical composition comprising a compound selected from Table 1, or a pharmaceutically acceptable form (e.g., salt) or an isotope derivative thereof, and a pharmaceutically acceptable excipient, carrier, or diluent, effective to treat or reduce one or more such symptoms, in a mammal, including a human.

In yet another aspect, the invention generally relates to a method for treating or reducing CLIFAHDD or other related neurological diseases that have overlapping symptoms to CLIFAHDD. The method comprises administering to a subject in need thereof a pharmaceutical composition comprising a compound selected from Table 1, or a pharmaceutically acceptable form (e.g., salt) or an isotope derivative thereof, and a pharmaceutically acceptable excipient, carrier, or diluent, effective to treat or reduce CLIFAHDD, or a related disease or condition, in a mammal, including a human.

In yet another aspect, the invention generally relates to a method for modifying a neuronal membrane property to achieve a change in its activity. The method comprises administering to a subject in need thereof a pharmaceutical composition comprising a compound selected from Table 1, or a pharmaceutically acceptable form (e.g., salt) or an isotope derivative thereof, and a pharmaceutically acceptable excipient, carrier, or diluent, effective to inhibit a neuronal activity, in a mammal, including a human.

In certain embodiment, the compound is nefopam. In certain embodiment, the compound is nefopam in the form of a hydrochloride salt.

In certain embodiment, the compound is amlodipine. In certain embodiment, the compound is amlodipine in the form of a besylate salt.

In certain embodiment, the compound is benzocaine.

In certain embodiment, the compound is berbamine. In certain embodiment, the compound is berbamine in the form of a dihydrochloride salt.

In certain embodiment, the compound is mexiletine. In certain embodiment, the compound is mexiletine in the form of a hydrochloride salt.

In certain embodiment, the compound is verapamil. In certain embodiment, the compound is verapamil in the form of a hydrochloride salt.

In certain embodiment, the method further comprises administering to a subject in need thereof a second, third, or further compound selected from Table 1 (i.e., two, three or more compounds selected from Table 1.)

In yet another aspect, the invention generally relates to a pharmaceutical composition comprising a therapeutically effective amount of a compound selected from Table 1, or a pharmaceutically acceptable form (e.g., salt) or an isotope derivative thereof, and a pharmaceutically acceptable excipient, carrier, or diluent.

In certain embodiment, the pharmaceutical composition of the invention suitable for oral administration. In certain embodiment, the compound(s) in the pharmaceutical composition is in the form of a salt.

In yet another aspect, the invention generally relates to a unit dosage form comprising the pharmaceutical composition of a compound selected from Table 1.

In certain embodiment, the unit dosage is a tablet. In certain embodiment, the unit dosage is a capsule.

In yet another aspect, the invention generally relates to use of a compound selected from Table 1, or a pharmaceutically acceptable form (e.g., salt) or an isotope derivative thereof, and a pharmaceutically acceptable excipient, carrier, or diluent, in the preparation of a medicament for treating or reducing CLIFAHDD or other related neurological diseases that have overlapping symptoms to CLIFAHDD.

In yet another aspect, the invention generally relates to use of a compound selected from Table 1, or a pharmaceutically acceptable form (e.g., salt) or an isotope derivative thereof, and a pharmaceutically acceptable excipient, carrier, or diluent, in the preparation of a medicament for treating or reducing one or more symptoms or diseases associated with NALCN R1181Q mutation.

In yet another aspect, the invention generally relates to use of a compound selected from Table 1, or a pharmaceutically acceptable form (e.g., salt) or an isotope derivative thereof, and a pharmaceutically acceptable excipient, carrier, or diluent, in the preparation of a medicament for treating or reducing CLIFAHDD and similar channel-mutation-related congenital disorders.

In yet another aspect, the invention generally relates to use of a compound selected from Table 1, or a pharmaceutically acceptable form (e.g., salt) or an isotope derivative thereof, and a pharmaceutically acceptable excipient, carrier, or diluent, in the preparation of a medicament for modifying a neuronal membrane property to achieve a change in its activity.

Certain compounds of the present invention may exist in particular geometric or stereoisomeric forms. The present invention contemplates all such compounds, including cis- and trans-isomers, R- and S-enantiomers, diastereomers, (d)-isomers, (l)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

Isomeric mixtures containing any of a variety of isomer ratios may be utilized in accordance with the present invention. For example, where only two isomers are combined, mixtures containing 50:50, 60:40, 70:30, 80:20, 90:10, 95:5, 96:4, 97:3, 98:2, 99:1, or 100:0 isomer ratios are contemplated by the present invention. Those of ordinary skill in the art will readily appreciate that analogous ratios are contemplated for more complex isomer mixtures.

If, for instance, a particular enantiomer of a compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic methods well known in the art, and subsequent recovery of the pure enantiomers.

Isotopically-labeled compounds are also within the scope of the present disclosure. As used herein, an “isotopically-labeled compound” refers to a presently disclosed compound including pharmaceutical salts and prodrugs thereof, each as described herein, in which one or more atoms are replaced by an atom having an atomic mass or mass number different from the atomic mass or mass number usually found in nature. Examples of isotopes that can be incorporated into compounds presently disclosed include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively.

By isotopically-labeling the presently disclosed compounds, the compounds may be useful in drug and/or substrate tissue distribution assays. Tritiated (³H) and carbon-14 (¹⁴C) labeled compounds are particularly preferred for their ease of preparation and detectability. Further, substitution with heavier isotopes such as deuterium (²H) can afford certain therapeutic advantages resulting from greater metabolic stability, for example increased in vivo half-life or reduced dosage requirements and, hence, may be preferred in some circumstances. Isotopically labeled compounds presently disclosed, including pharmaceutical salts, esters, and prodrugs thereof, can be prepared by any means known in the art.

Further, substitution of normally abundant hydrogen (¹H) with heavier isotopes such as deuterium can afford certain therapeutic advantages, e.g., resulting from improved absorption, distribution, metabolism and/or excretion (ADME) properties, creating drugs with improved efficacy, safety, and/or tolerability. Benefits may also be obtained from replacement of normally abundant ¹²C with ¹³C. (See, WO 2007/005643, WO 2007/005644, WO 2007/016361, and WO 2007/016431.)

Stereoisomers (e.g., cis and trans isomers) and all optical isomers of a presently disclosed compound (e.g., R and S enantiomers), as well as racemic, diastereomeric and other mixtures of such isomers are within the scope of the present disclosure.

Compounds of the present invention are, subsequent to their preparation, preferably isolated and purified to obtain a composition containing an amount by weight equal to or greater than 95% (“substantially pure”), which is then used or formulated as described herein. In certain embodiments, the compounds of the present invention are more than 99% pure.

Solvates and polymorphs of the compounds of the invention are also contemplated herein. Solvates of the compounds of the present invention include, for example, hydrates.

Any appropriate route of administration can be employed, for example, parenteral, intravenous, subcutaneous, intramuscular, intraventricular, intracorporeal, intraperitoneal, rectal, or oral administration. Most suitable means of administration for a particular patient will depend on the nature and severity of the disease or condition being treated or the nature of the therapy being used and on the nature of the active compound.

Compositions for parenteral injection comprise pharmaceutically-acceptable sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol, and the like), carboxymethylcellulose and suitable mixtures thereof, vegetable oils (such as olive oil), and injectable organic esters such as ethyl oleate. Proper fluidity may be maintained, for example, by the use of coating materials such as lecithin, by the maintenance of the required particle size in the case of dispersions, and by the use of surfactants.

These compositions can also contain adjuvants such as preservative, wetting agents, emulsifying agents, and dispersing agents. Prevention of the action of microorganisms may be ensured by the inclusion of various antibacterial and antifungal agents, for example, paragen, chlorobutanol, phenol sorbic acid, and the like. It may also be desirable to include isotonic agents such as sugars, sodium chloride, and the like. Prolonged absorption of the injectable pharmaceutical form may be brought about by the inclusion of agents which delay absorption, such as aluminum monostearate and gelatin.

Compounds of the present invention may also be administered in the form of liposomes. As is known in the art, liposomes are generally derived from phospholipids or other lipid substances. Liposomes are formed by mono- or multi-lamellar hydrated liquid crystals that are dispersed in an aqueous medium. Any non-toxic, physiologically-acceptable and metabolizable lipid capable of forming liposomes can be used. The present compositions in liposome form can contain, in addition to a compound of the present invention, stabilizers, preservatives, excipients, and the like. The preferred lipids are the phospholipids and the phosphatidyl cholines (lecithins), both natural and synthetic. Methods to form liposomes are known in the art. See, for example, Prescott, Ed., Methods in Cell Biology, Volume XIV, Academic Press, New York, N.Y. (1976), p. 33 et seq.

Total daily dose of the compositions of the invention to be administered to a human or other mammal host in single or divided doses may be in amounts, for example, from 0.0001 to 300 mg/kg body weight daily and more usually 1 to 300 mg/kg body weight. The dose, from 0.0001 to 300 mg/kg body, may be given twice a day.

Solid dosage forms for oral administration include capsules, tablets, pills, powders, and granules. In such solid dosage forms, the compounds described herein or derivatives thereof are admixed with at least one inert customary excipient (or carrier) such as sodium citrate or dicalcium phosphate or (i) fillers or extenders, as for example, starches, lactose, sucrose, glucose, mannitol, and silicic acid, (ii) binders, as for example, carboxymethylcellulose, alignates, gelatin, polyvinylpyrrolidone, sucrose, and acacia, (iii) humectants, as for example, glycerol, (iv) disintegrating agents, as for example, agar-agar, calcium carbonate, potato or tapioca starch, alginic acid, certain complex silicates, and sodium carbonate, (v) solution retarders, as for example, paraffin, (vi) absorption accelerators, as for example, quaternary ammonium compounds, (vii) wetting agents, as for example, cetyl alcohol, and glycerol monostearate, (viii) adsorbents, as for example, kaolin and bentonite, and (ix) lubricants, as for example, talc, calcium stearate, magnesium stearate, solid polyethylene glycols, sodium lauryl sulfate, or mixtures thereof. In the case of capsules, tablets, and pills, the dosage forms may also comprise buffering agents. Solid compositions of a similar type may also be employed as fillers in soft and hard-filled gelatin capsules using such excipients as lactose or milk sugar as well as high molecular weight polyethyleneglycols, and the like. Solid dosage forms such as tablets, dragees, capsules, pills, and granules can be prepared with coatings and shells, such as enteric coatings and others known in the art.

Liquid dosage forms for oral administration include pharmaceutically acceptable emulsions, solutions, suspensions, syrups, and elixirs. In addition to the active compounds, the liquid dosage forms may contain inert diluents commonly used in the art, such as water or other solvents, solubilizing agents, and emulsifiers, such as for example, ethyl alcohol, isopropyl alcohol, ethyl carbonate, ethyl acetate, benzyl alcohol, benzyl benzoate, propyleneglycol, 1,3-butyleneglycol, dimethylformamide, oils, in particular, cottonseed oil, groundnut oil, corn germ oil, olive oil, castor oil, sesame oil, glycerol, tetrahydrofurfuryl alcohol, polyethyleneglycols, and fatty acid esters of sorbitan, or mixtures of these substances, and the like. Besides such inert diluents, the composition can also include additional agents, such as wetting, emulsifying, suspending, sweetening, flavoring, or perfuming agents.

Materials, compositions, and components disclosed herein can be used for, can be used in conjunction with, can be used in preparation for, or are products of the disclosed methods and compositions. It is understood that when combinations, subsets, interactions, groups, etc. of these materials are disclosed that while specific reference of each various individual and collective combinations and permutations of these compounds may not be explicitly disclosed, each is specifically contemplated and described herein. For example, if a method is disclosed and discussed and a number of modifications that can be made to a number of molecules including in the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

### EXAMPLES

The below Examples describe certain exemplary embodiments of compounds prepared according to the disclosed invention. It will be appreciated that the following general methods, and other methods known to one of ordinary skill in the art, can be applied to compounds and subclasses and species thereof, as disclosed herein.

### Example 1. Screen of Putative NALCN Antagonists Using the Motor Behavior of the C. elegans NALCN(gf) Models

All experiments are performed on N2 non-transgenic worms and EG9152 GOF worms.

Control experiments were performed to assess what concentrations of DMSO (a vehicle used in this screen) can be tolerated by the N2 and GOF worms. Both can tolerate 2.5% DMSO in agar for multiple hours. Above this concentration, worms will die within minutes.

The ability of worms to tolerate agar with no additives (ex. cholesterol, peptone, KPO₄, CaCl₂), MgSO₄). Worms appeared healthy and displayed no behavioral defects within 1 hour on basic agar.

### Preparation of Compound Stock Solutions

Compounds were dissolved in the vehicle in which they were the most soluble (DMSO or molecular grade water). To make stock solutions, each compound was dissolved to its maximum solubility (this information can be found on the production company's website or on the excel sheet containing compound information).

Compounds were initially dissolved by vortexing. If they could not be dissolved, they were incubated in a 37° C. water bath with periodic vortexing (most compounds will dissolve at this point, usually within an hour).

Aliquots with the amount of compound to be mixed with media in each trial (25 μL if dissolved in DMSO and 100 μL if dissolved in water) were prepared to avoid exposing the compounds to multiple freeze-thaw cycles.

Most compounds were stored at −20° C. (as recommended by the production company). A few were stored at 4° C.

### Preparation of Screening Plates

Prepare agar: Multicell Bacteriological Grade Agar1 0.7-2.0 g/100 mL dH₂0. Microwave to dissolve (note: this will boil over rapidly).

Allow agar to cool to <50° C. Check with thermometer.

Target Mol compounds are synthesized at temperatures of 50-80° C. They must be exposed to temperatures no higher than 50° C. to prevent altering the compound. Agar sets from 32-40° C. You must work quickly to fill the plates before the agar solidifies.

The screen is performed in a 24-well flat-bottom plate. Add the desired amount of compound dissolved in either DMSO (max 25 μL) or water (max 100 μL) to the empty well first. Proceed to next step quickly.

Add the corresponding amount of molten agar to the well to reach 1 mL total (ex. 975 μL agar to 25 μL of compound). Immediately begin mixing with pipette tip until the compound dissolves (at this point the mixture should be clear).

Fill the remainder of the plate (I usually prepare duplicate wells for each compound) and cover with aluminum foil to protect the compounds from light. Leave at room temperature overnight to allow the plate to dry before screening.

### Preparation of Worm Populations

Pick adult worms to fresh plates 3-4 d before conducting the screen. Prepare at least 4 small plates for each line. Only L4 worms can be used in the screen. Ensure that N2 control lines are also prepared.

### Performing a Screen

Pick a line to start with. Place at least 10 L4 worms in each well. Start timing as soon as a well has been filled.

Observe the behavior of the worms after 20 mins and 40 mins of compound exposure. Touch the tail of each worm gently with a platinum wire to promote motion. Record any observations. (I like to record the proportion of worms that retain the original phenotype, the proportion that show full body paralysis/death, and the proportion that show paralysis of the neck only).

When the screen is finished, remove worms from each well using hot platinum wire.

Proceed to screen the next line.

Exemplary results of screening are provided in Table 1.

TABLE 1
Exemplary Results from Compound Screening

| Compound | | Trial 1 | Trial 2 | Trial 3 | Trial 4 |
|---|---|---|---|---|---|
| Amlodipine | Concentration | 2.5 mM | 2.5 mM | 2.5 mM | 1.25 mM |
| Besylate | | (18.11.28) | (18.12.11) | (18.12.11) | (18.12.11) |
| | GOF | 30 mins: Most | 30 mins: 42% | 30 mins: 33% | 30 mins: 64% |
| | | show rescue | rescue, 25% | rescue, 11: GOF, | rescue, 36% GOF. |
| | | phenotype, no | GOF, 33% LOF | 55% LOF. | 1 hr: 64% rescue, |
| | | motility without | 1 hr: 10% | 1 hr: 100% LOF | 27% GOF, 9% |
| | | stimulation | rescue, 90% | | LOF. |
| | | | LOF | | |
| | N2 | 30 mins: 10% | 30 mins: 22% | 30 mins: 22% | 30 mins: 50% |
| | | “rescue,” 10% | “rescue,” 78% | “rescue,” 78% | “rescue” (slight |
| | | WT(N2), 80% | LOF. | LOF. | but present), 50% |
| | | LOF. | 1 hr: 100% | 1 hr: 100% LOF | WT(N2). |
| | | | LOF | | 1 hr: 90% rescue, |
| | | | | | 10% WT(N2). |
| Berbamine | Concentration | 4.4 mM | 4.4 mM | 4.4 mM | 2.2 mM |
| dihydrochloride | | (18.11.15) | (18.11.28) | (18.12.11) | (18.12.11) |
| | GOF | 30 min: 20% | 30 min: 70% | 30 min: 33% | 30 min: 20% |
| | | rescue, 80% | rescue, 30% | rescue, 42% | rescue, 80% GOF. |
| | | GOF. | GOF. | GOF, 25% LOF. | 1 hr: no change. |
| | | 1 hr: 50-60% | 1 hr: no change. | 1 hr: no change. | |
| | | rescue, 50-40% | | | |
| | | GOF. | | | |
| | N2 | 30 min: No | 30 min: 70% | 30 min: 30% | 30 min: 10% |
| | | response | “rescue,” 30% | “rescue,” 70% | “rescue,” 90% |
| | | | WT(N2). | WT(N2). | WT(N2). |
| | | | | 1 hr: no change. | 1 hr: no change. |
| Benzocaine | Concentration | 4.71 mM | 4.71 mM | 2.4 mM | 2.4 mM |
| | | (19.01.15) | (19.01.15) | (19.01.23) | (19.01.23) |
| | GOF | 30 min: 38% | 30 min: 8% | 20 min: 40% | 20 min: 33% |
| | Show | rescue, 62% | rescue, 92% | rescue, 47% | rescue, 33% GOF, |
| | paralysis of | LOF. Rescue | LOF. | GOF, 13% LOF. | 33% LOF. |
| | neck and | worms still | 1 hr: 100% | 40 min: 8% | 40 min: 11% |
| | reduced body | move fairly | LOF. | rescue, 42% | rescue, 55% GOF, |
| | bend | quickly. | | GOF, 50% LOF. | 34% LOF. |
| | amplitude but | Reduced | | | |
| | not as | amplitude of | | | |
| | obvious a | body bends | | | |
| | phenotype as | overall. | | | |
| | Verapamil. | 1 hr: 100% | | | |
| | | LOF. | | | |
| | N2 | 30 min: 100% | 30 min: 18% | 20 min: 28% N2, | 20 min: 27% N2, |
| | | LOF. | sluggish N2, | 72% LOF | 73% LOF. |
| | | 1 hr: 100% | 82% LOF. | 40 min: 28% N2, | 40 min: 7% N2 |
| | | LOF. | 1 hr: 100% | 72% LOF. | (sluggish), 93% |
| | | | LOF. | | LOF. |
| Mexiletine | Concentration | 19.95 mM | 10 mM | 5.0 mM | 2.5 mM |
| | | (19.01.16) | (19.01.16) | (19.01.23) | (19.01.23) |
| | GOF | 20 min: 100% | 20 min: 20% | 20 min: 36% | 20 min: 82% |
| | | LOF. | GOF, 80% | rescue (slight), | GOF, 18% LOF. |
| | | 50 min: 100% | LOF. | 64% GOF. | 40 min: 11% |
| | | LOF. | 50 min: 100% | 40 min: 50% | rescue (rest in |
| | | | LOF. | rescue (rest in | coils similar to |
| | | | | coils similar to | Tolperisone and |
| | | | | Tolperisone and | are relatively |
| | | | | are relatively | unresponsive to |
| | | | | unresponsive to | touch although |
| | | | | touch although | they can move), |
| | | | | they can move), | 67% GOF, 22% |
| | | | | 29% GOF, 21% | LOF. |
| | | | | LOF. | |
| | N2 | 20 min: 14% | 20 min: 64% | 20 min: 20% | 20 min: 17% |
| | | N2, 86% LOF. | N2, 36% LOF. | “rescue,” 10% | “rescue,” 58% |
| | | 50 min: 100% | 50 min: 9% | GOF, 70% LOF. | N2, 25% LOF. |
| | | LOF. | N2, 91% LOF. | 40 min: 10% N2, | 40 min: 64% |
| | | | | 90% LOF. | “rescue,” 18% |
| | | | | | N2, 18% LOF. |
| Nefopam | Concentration | 6.65 mM | 6.65 mM | 3.3 mM | 3.3 mM |
| | | (19.01.16) | (19.01.16) | (19.02.6) | (19.02.6) |
| | GOF | 20 min: 33% | 20 min: 90% | 20 min: 70% | 20 min: 69% |
| | | rescue, 66% | rescue, 10% | rescue, 30% | rescue, 31% GOF. |
| | | LOF. | LOF. | LOF. | 40 min: 100% |
| | | 50 min: 100% | 50 min: 60% | 40 min: 25% | rescue (sluggish) |
| | | LOF. | rescue, 40% | rescue, 8% GOF, | |
| | | | LOF. | 67% LOF. | |
| | N2 | 20 min: 9% | 20 min: 23% | 20 min: 15% | 20 min: 7% |
| | | “rescue,” 91% | “rescue,” 77% | “rescue,” 23% | “rescue,” 43% |
| | | LOF. | LOF | N2, 62% LOF. | N2, 50% LOF. |
| | | 50 min: 100% | 50 min: 100% | | |
| | | LOF. | LOF. | | |
| Verapamil | Concentration | 10 mM | 10 mM | 10 mM | |
| | | (18.11.28) | (18.12.11) | | |
| | GOF | 30 min: 60-70% | 30 min: 67% | 30 min: 5% | |
| | | rescue, | rescue, 11% | rescue, 88% | |
| | | 1 hr: most LOF | GOF, 22% | GOF, 7% LOF | |
| | | | LOF. | 1 hr: 20% rescue, | |
| | | | 1 hr: 10% | 72% GOF, 8% | |
| | | | GOF, 90% | LOF. | |
| | | | LOF. | | |
| | N2 | 30 min: fewer | 30 min: 38% | 30 min: 6% | |
| | | worms show | WT(N2), 62% | “rescue,” 94% | |
| | | “rescue” 1 hr: | LOF. | WT(N2). | |
| | | most paralyzed. | 1 hr: 10% | 1 hr: 6% | |
| | | | WT(N2), 90% | “rescue,” 94% | |
| | | | LOF. | WT(N2). | |

### Example 2. Electrophysiology Tests

Briefly, the cerebral cortex was isolated from embryos of E16 pregnant mouse (C57/BL6) and cut into small cubes. After being digested in 0.05% Trypsin for 25-30 mins in 37° C. in incubator, the Trypsin was neutralized with 1-2 ml DMEM (containing 10% FBS. The pellets were then triturated with a pipette for 10-15 time and then diluted into plating media (DMEM with 10% FBS). The cell suspension was sieved through a 70 mm nylon filter and plated onto cover slips coated with poly-D-lysine (PDL) in 35 mm dishes at density of 2×105 cells per dish. After culturing for 2 hrs in incubator, the plating media was replaced with the following media: Neurobasal media supplemented with 2% B27. Neurobasal media was changed every 3 days until the experiment start.

Membrane potentials of neurons were recorded in whole-cell configuration at 20° C.-22° C. in a mouse E16 embryonic cortical neuron preparation. The pipette solution contained (in mM): K-Gluconate 115; KCl 25; CaCl₂) 0.1; MgCl₂ 5; BAPTA 1; HEPES 10; Na₂ATP 5; Na₂GTP 0.5; cAMP 0.5; cGMP 0.5, pH 7.2 with KOH, 320 mOsm. The bath solution consisted of (in mM): NaCl 150; KCl 5; CaCl₂) 5; MgCl₂ 1; glucose 10; sucrose 5; HEPES 15, pH 7.3 with NaOH, 330 mOsm. For zero and 15 Na+ solution, extracellular Na+ ([Na+]o) was replaced with N-methyl-D-glucamine (NMDG+) or Tris+. RMP was recorded at 0 pA. Healthy preparations were selected based on following criteria: whole-cell capacitance (1-2.2 pF), steady state leak current (−40 to 0 pA at −60 mV) and RMP (−40 to −15 mV, at 150 mM Na+).

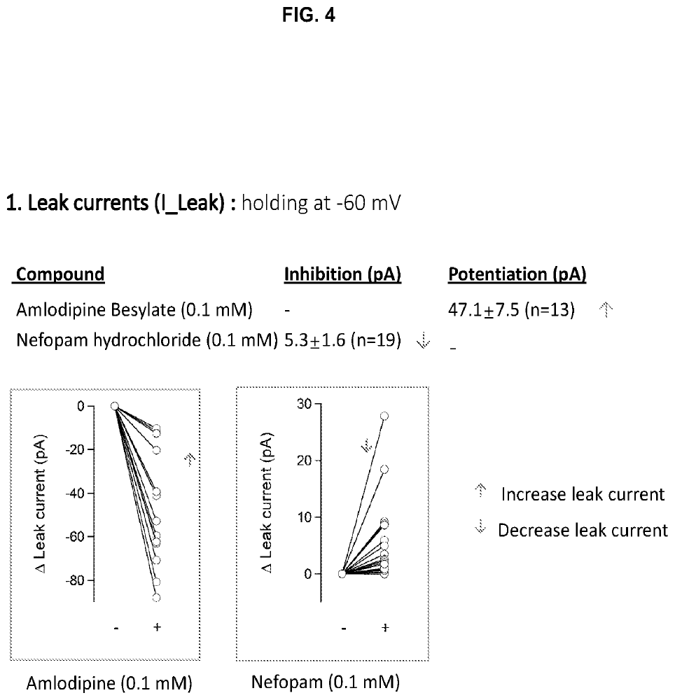

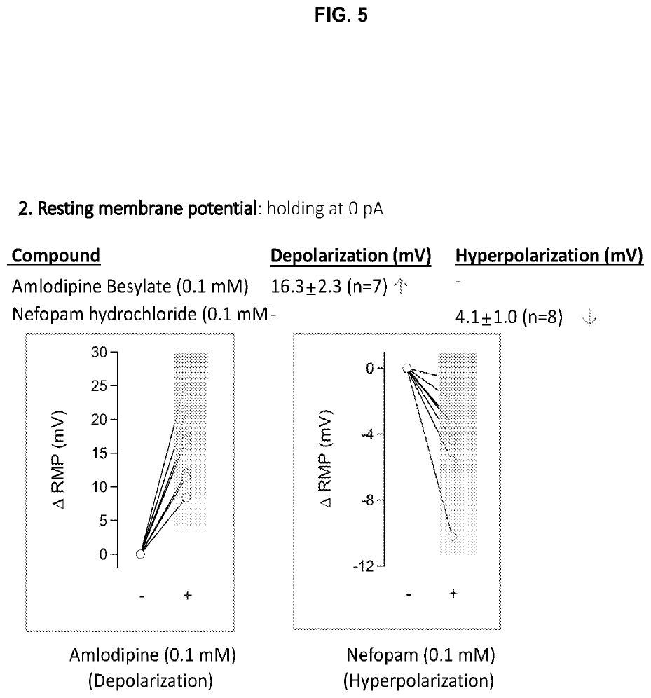

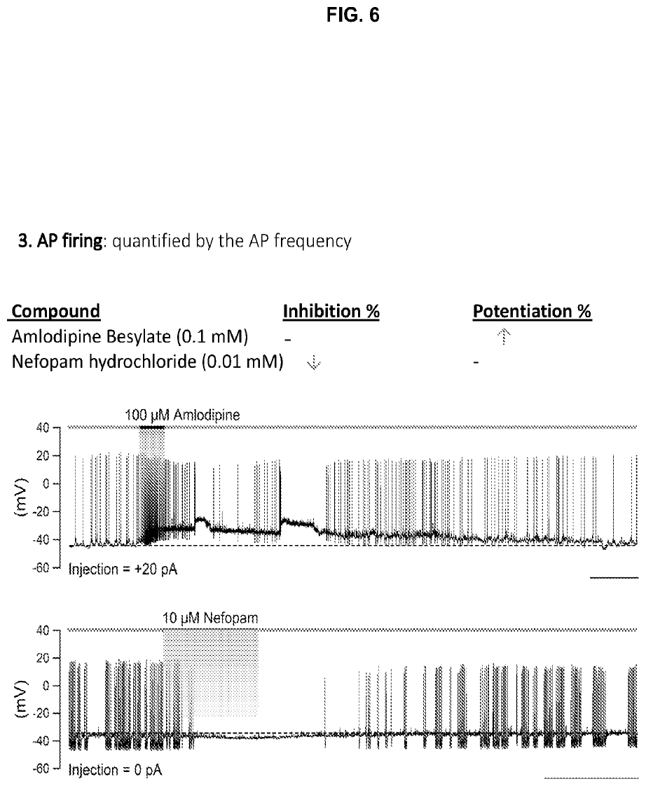

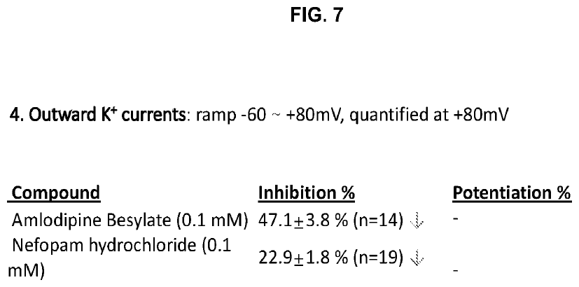

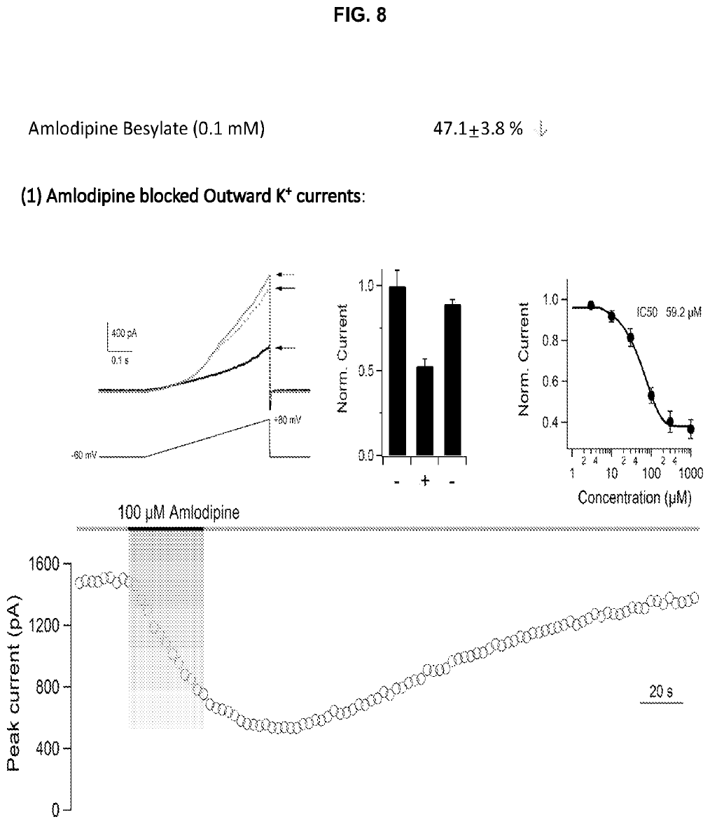

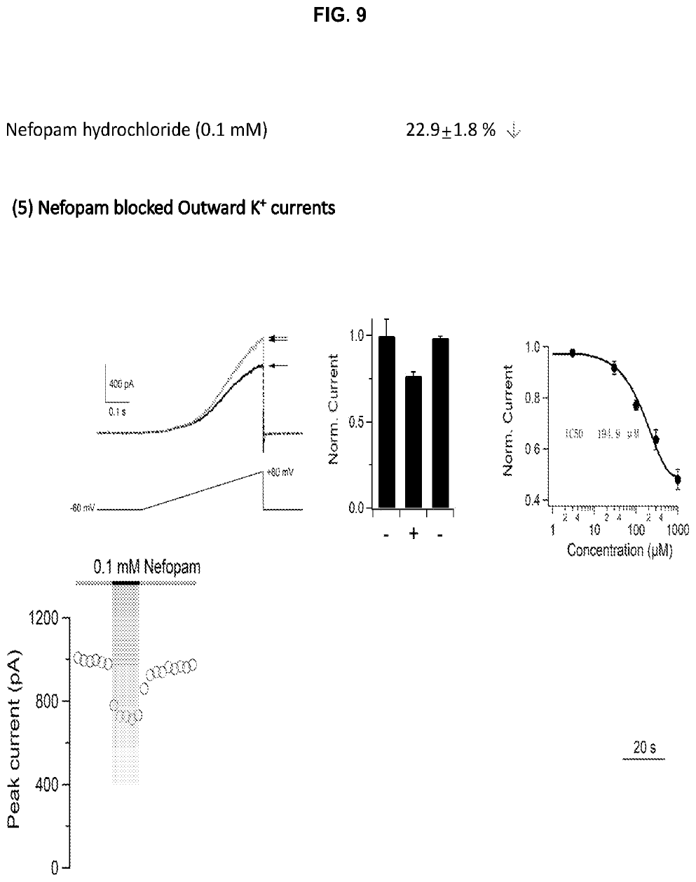

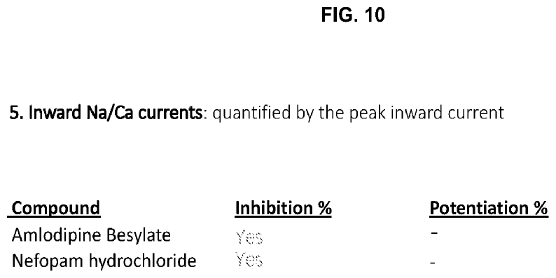

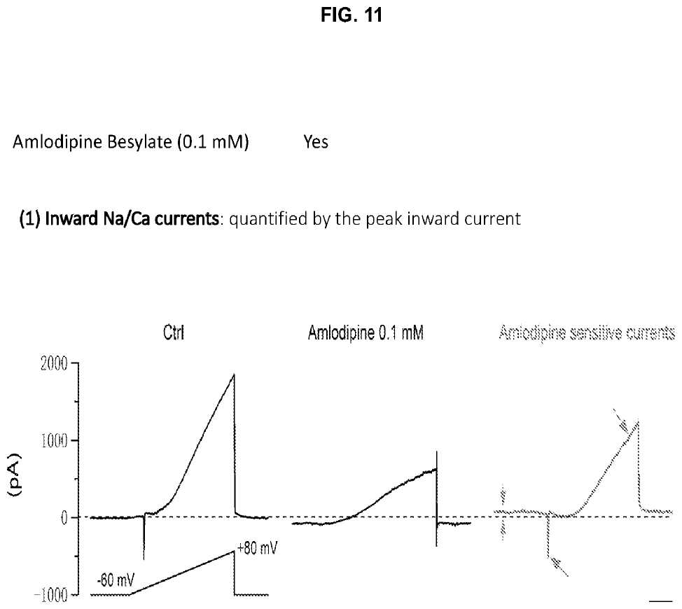

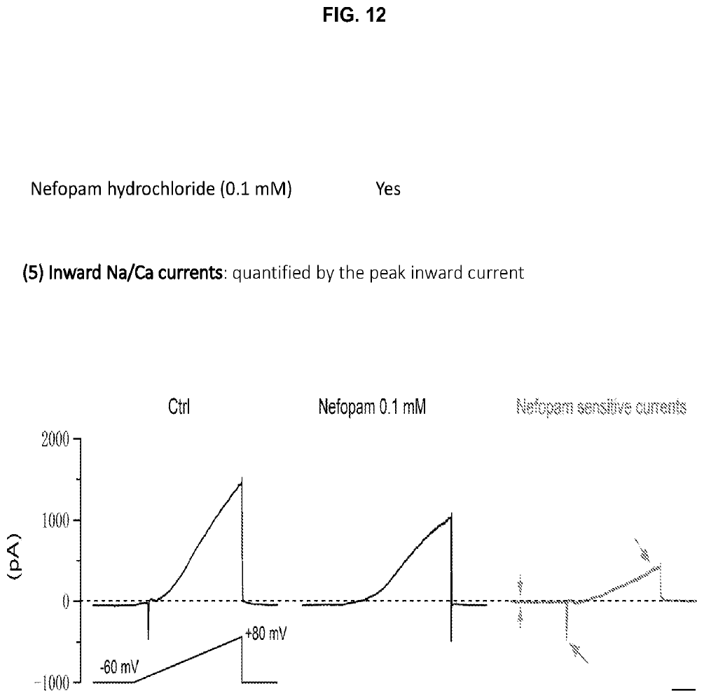

Exemplary data is shown in FIGS. 4-12.

The term “comprising”, when used to define compositions and methods, is intended to mean that the compositions and methods include the recited elements, but do not exclude other elements. The term “consisting essentially of”, when used to define compositions and methods, shall mean that the compositions and methods include the recited elements and exclude other elements of any essential significance to the compositions and methods. For example, “consisting essentially of” refers to administration of the pharmacologically active agents expressly recited and excludes pharmacologically active agents not expressly recited. The term “consisting essentially of” does not exclude pharmacologically inactive or inert agents, e.g., pharmaceutically acceptable excipients, carriers or diluents. The term “consisting of” shall mean excluding trace elements of other ingredients and substantial method steps. Embodiments defined by each of these transition terms are within the scope of this invention.

Applicant's disclosure is described herein in preferred embodiments with reference to the Figures, in which like numbers represent the same or similar elements. Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

The described features, structures, or characteristics of Applicant's disclosure may be combined in any suitable manner in one or more embodiments. In the description, herein, numerous specific details are recited to provide a thorough understanding of embodiments of the invention. One skilled in the relevant art will recognize, however, that Applicant's composition and/or method may be practiced without one or more of the specific details, or with other methods, components, materials, and so forth. In other instances, well-known structures, materials, or operations are not shown or described in detail to avoid obscuring aspects of the disclosure.

Unless defined otherwise, all technical and scientific terms used herein have the same meaning as commonly understood by one of ordinary skill in the art. Although any methods and materials similar or equivalent to those described herein can also be used in the practice or testing of the present disclosure, the preferred methods and materials are now described. Methods recited herein may be carried out in any order that is logically possible, in addition to a particular order disclosed.

### INCORPORATION BY REFERENCE

References and citations to other documents, such as patents, patent applications, patent publications, journals, books, papers, web contents, have been made in this disclosure. All such documents are hereby incorporated herein by reference in their entirety for all purposes. Any material, or portion thereof, that is said to be incorporated by reference herein, but which conflicts with existing definitions, statements, or other disclosure material explicitly set forth herein is only incorporated to the extent that no conflict arises between that incorporated material and the present disclosure material. In the event of a conflict, the conflict is to be resolved in favor of the present disclosure as the preferred disclosure.

### EQUIVALENTS

The representative examples are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples and the references to the scientific and patent literature included herein. The examples contain important additional information, exemplification and guidance that can be adapted to the practice of this invention in its various embodiments and equivalents thereof.

1. A method for treating or reducing a congenital disorder, comprising administering to a subject in need thereof a pharmaceutical composition comprising a compound selected from the group consisting of: 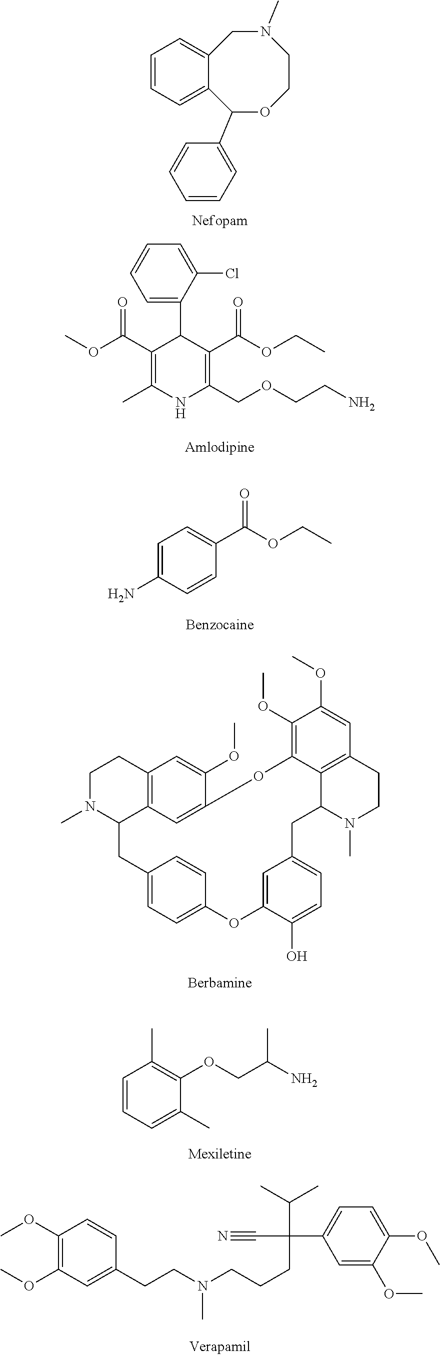
or a pharmaceutically acceptable form or an isotope derivative thereof, and a pharmaceutically acceptable excipient, carrier, or diluent, effective to treat or reduce a congenital disorder, or a related disease or condition, in a mammal, including a human.

2. A method for treating or reducing one or more symptoms or diseases associated with NALCN caused by any Gain-of-Function or Loss-of Function gene mutations, comprising administering to a subject in need thereof a pharmaceutical composition comprising a compound selected from the group consisting of: 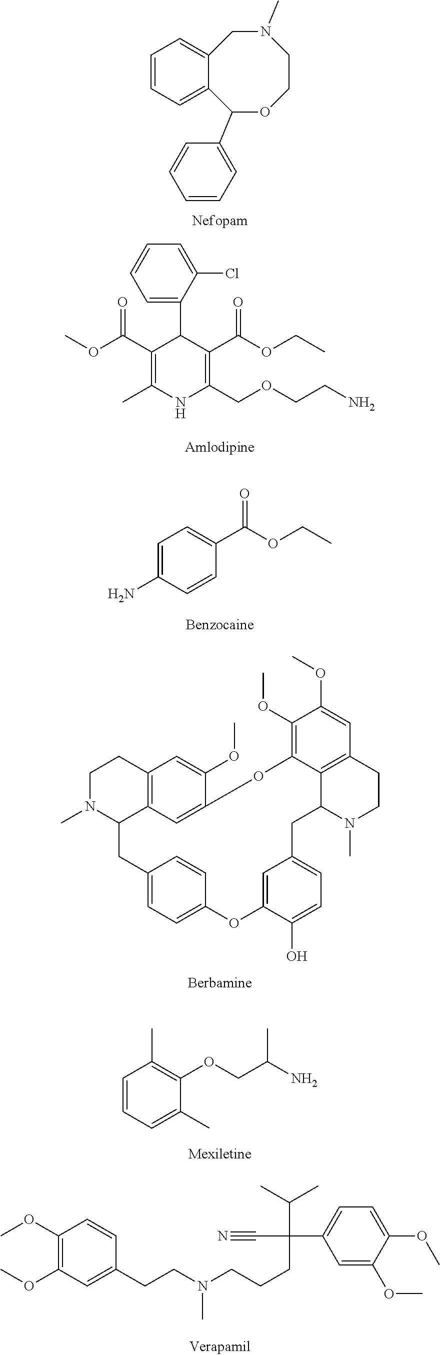
or a pharmaceutically acceptable form or an isotope derivative thereof, and a pharmaceutically acceptable excipient, carrier, or diluent, effective to treat or reduce one or more such symptoms, in a mammal, including a human.

3. The method of claim 2, wherein the one or more symptoms or diseases is associated with NALCN R1181Q mutation

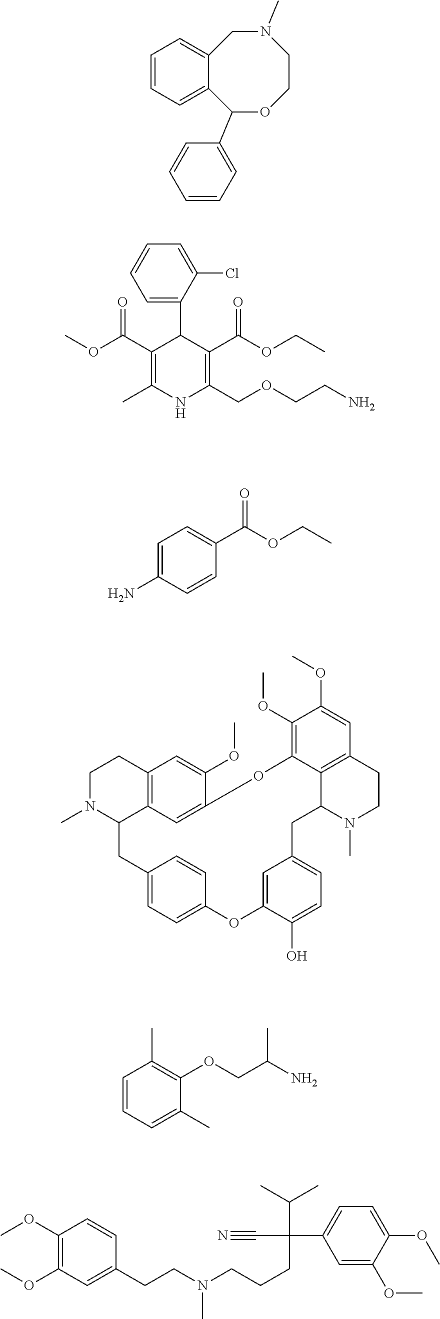

4. The method of claim 1, wherein the congenital disorder is congenital contractures of the limbs and face, hypotonia, and developmental delay (CLIFAHDD), or other related neurological diseases that have overlapping symptoms to CLIFAHDD

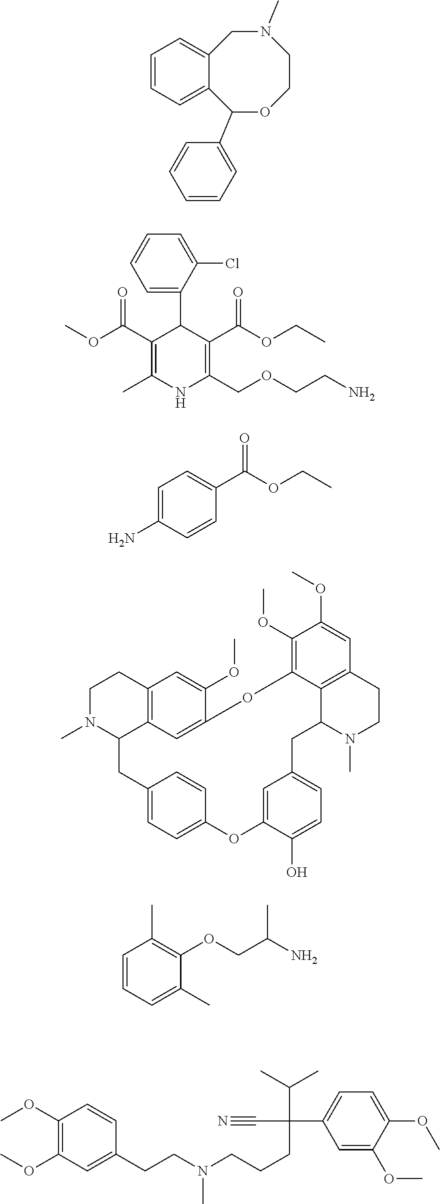

5. (canceled)

6. The method of claim 1, wherein the compound is nefopam.

7. The method of claim 6, wherein the compound is in the form of a hydrochloride salt.

8. The method of claim 1, wherein the compound is amlodipine.

9. The method of claim 8, wherein the compound is in the form of a besylate salt.

10. The method of claim 1, wherein the compound is benzocaine.

11. The method of claim 1, wherein the compound is berbamine.

12. The method of claim 11, wherein the compound is in the form of a dihydrochloride salt.

13. The method of claim 1, wherein the compound is mexiletine.

14. The method of claim 13, wherein the compound is in the form of a hydrochloride salt.

15. The method of claim 1, wherein the compound is verapamil.

16. The method of claim 15, wherein the compound is in the form of a hydrochloride salt.

17. The method of claim 1, further comprising administering to a subject in need thereof a second compound selected from the group of compounds.

18. The method of claim 17, further comprising administering to a subject in need thereof a third or further compound selected from the group of compounds.

19. A pharmaceutical composition comprising a therapeutically effective amount of a compound selected from the group consisting of: 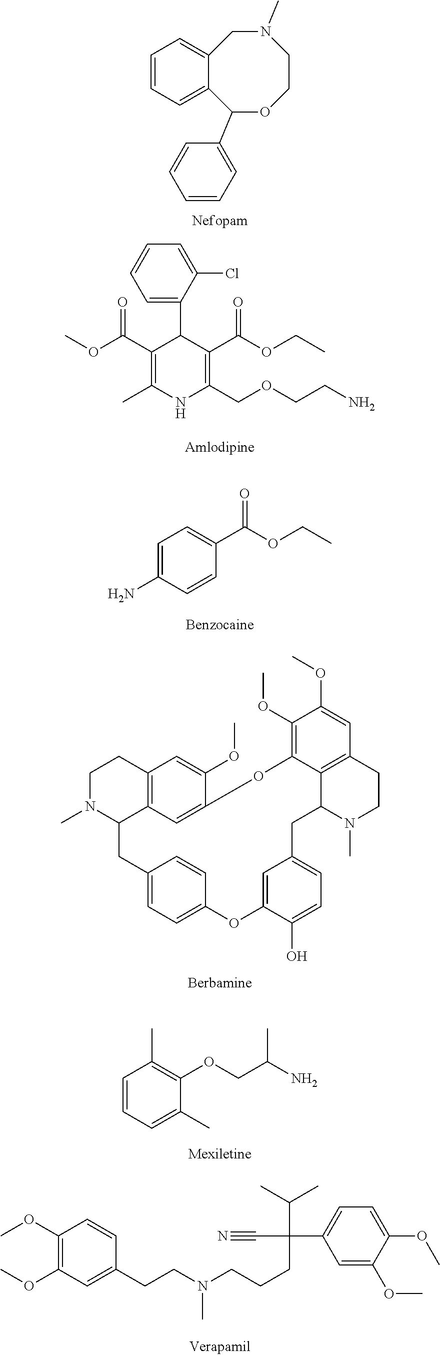
or a pharmaceutically acceptable form or an isotope derivative thereof, and a pharmaceutically acceptable excipient, carrier, or diluent.

20. The pharmaceutical composition of claim 19, being suitable for oral administration.

21. (canceled)

22. A unit dosage form comprising the pharmaceutical composition of claim 19.

23-29. (canceled)
